(19) United States
(12) Patent Application Publication
(10) Pub. No.: US 2005/0272805 A1
(43) Pub. Date: Dec. 8, 2005

(54) Treatment of gastrointestinal disorders with duloxetine

(76) Inventors: Michael J. Detke, Carnmel, IN (US); David Joel Goldstein, Indianapolis, IN (US); Smriti Iyengar, Carmel, IN (US)

Correspondence Address: ELI LILLY AND COMPANY;PATENT DIVISION, P.O. BOX 6288, INDIANAPOLIS, IN, 46206-6288, US

(21) Appl. No.: 10/533,214

(22) PCT Filed: Nov. 18, 2003
(86) PCT No.: PCT/US03/35051

§ 371 Date: Apr. 28, 2005

Related U.S. Application Data
(60) Provisional application No. 60/427,514, filed on Nov. 19, 2002.

(51) Int. Cl. A61K 31/381

(52) U.S. Cl. 514/438

(57) ABSTRACT

The present invention belongs to the fields of medicinal and pharmaceutical chemistry, and provides a new method of treating gastrointestinal disorders by the administration of duloxetine.

The present invention belongs to the fields of medicinal and pharmaceutical chemistry, and provides a new method of treating gastrointestinal disorders by administration of duloxetine.

For some years, it has been recognized that the chemistry of serotonin and norepinephrine are extremely important in neurological processes, and pharmacologists and medical researchers have been very actively studying the mechanisms of those neurotransmitters in the brain. Concomitantly, the synthesis and study of pharmaceuticals which affect serotonin and norepinephrine processes in the brain are of great interest and are also being intensively studied, both by pharmaceutical chemists and by medical researchers as well.

Duloxetine inhibits the reuptake of both serotonin and norepinephrine, and is now in clinical trials as an antidepressant drug, and also for the treatment of urinary Incontinence, diabetic neuropathic pain and fibromyalgia. The present invention provides the use of duloxetine for an additional important purpose, the treatment of gastrointestinal disorders.

Commonly encountered gastrointestinal disorders include inflammatory bowel disorders (IBD) and functional bowel disorders (FBD), including dyspepsia. These GI disorders include a wide range of disease states that are currently only moderately controlled, including Crohn's disease, ileitis, ischemic bowel disease, and ulcerative colitis, as well as IBD, irritable bowel syndrome, dyspepsia, gastro-esophageal reflux, FBD, and other forms of visceral pain (and/or GI tract dysfunction).

The present invention provides a method of treating a gastrointestinal disorder in a patient comprising administering to the patient an effective amount of duloxetine.

Duloxetine is N-methyl-3-(1-naphthalenyloxy)-3-(2-thienyl)propanamine. It is usually administered as the (+) enantiomer, and as the hydrochloride salt. It was first taught by U.S. Pat. No. 4,956,388, which teaches the synthesis of the compound as well as its high potency as an uptake inhibitor of both serotonin and norepinephrine. The word “duloxetine” will be used here to refer to any acid addition salt or the free base of the molecule, as well as to either an enantiomer or the racemate. It is to be understood, however, that the (+) enantiomer is preferred.

The most preferred dose of duloxetine for the treatment of a given patient with any particular gastrointestinal disorder may vary, depending on the characteristics of the patient, as all clinicians and medical doctors are aware. Factors such as other diseases from which the patient suffers, the patient's age and size, and other medications which the patient may be using will have an effect on the duloxetine dose and will be taken into account. In general, however, the daily dose of duloxetine is from about 1 to about 120 mg. The most preferred dose range is between 60 mg QD and 80 mg per day.

Duloxetine is orally available and presently is orally administered, in the form of a capsule full of enteric coated granules. Oral administration in such forms is preferred in the practice of the present invention. However, other routes of administration are also practical and may be preferred in certain cases. For example, transdermal administration may be very desirable for patients who are forgetful or petulant about taking oral medicine.

In general, the formulation of duloxetine for use in the present invention follows the methods used in formulating duloxetine for other purposes, and indeed methods usual in pharmaceutical science are appropriate. However, a preferred formulation of duloxetine comprises enteric pellets, or granules, of which a number are charged in a gelatin capsule. The duloxetine enteric pellet formulation is described in U.S. Pat. No. 5,508,276.

The patient to be benefited by practice of the present invention is a patient having one or more of the gastrointestinal disorders discussed below. Diagnosis of these disorders, or the identification of a patient at risk of one or more of them, is to be made by a physician. It is presently believed that duloxetine's potency in inhibiting the uptake of serotonin and norepinephrine is the mechanism by which it benefits such patients, by alleviating the effects of the disorder from which the patient suffers, or even eliminating the disorder completely.

The disorders which are treated or prevented in the practice of the present invention may be described as follows:
inflammatory bowel disorders (IBD),
functional bowel disorders (FBD),
dyspepsia,
crohn's disease,
iletis,
ischemic bowel disease,
ulcerative colitis,
irritable bowel syndrome, and
gastroesophageal reflux.

Inflammatory bowel disorders include, but are not limited to, crohn's disease and ulcerative colitis.

One such type of patient who would benefit from this invention would be a patient suffering from IBS. Such a patient would have symptoms characterized by “ROME II Criteria”. See practitioner 217:276-280 (1976. Symptoms include but are not limited to intermittent diarrhea, discomfort, abdominal pain and/or constipation or bloating. Upon administration of duloxetine a patient will experience alleviation of symptoms associated with IBS.

As used herein the term “effective amount” refers to the amount or dose of the compound, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances. For example, a typical daily dose may contain from about 25 mg to about 300 mg of the active ingredient. Duloxetine can be administered by a variety of routes including oral, rectal, transdermal, subcutaneous, intravenous, intramuscular, bucal or intranasal routes. Alternatively, the compound may be administered by continuous infusion.

As used herein the term “patient” refers to a mammal, such as a mouse, guinea pig, rat, dog or human. It is understood that the preferred patient is a human.

The term “treating” (or “treat”) as used herein includes its generally accepted meaning which encompasses prohibiting, preventing, restraining, and slowing, stopping, or reversing progression of a resultant symptom or desired process. As such, the methods of this invention encompass both therapeutic and prophylactic administration.

The compound of the present invention is preferably formulated prior to administration. Therefore, another aspect of the present invention is a pharmaceutical formulation comprising a compound of formula I, a pharmaceutically acceptable metabolically labile ester thereof, or a pharmaceutically acceptable salt thereof, and a pharmaceutically-acceptable carrier, diluent, or excipient. The pharmaceutical formulations may be prepared by using procedures well-known by one of ordinary skill in the art. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragacanth, gelatin, calcium silicate, microcrystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

As used herein, the term “active ingredient” refers to duloxetine.

The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient.

The ability of duloxetine to treat gastrointestinal diseases according to this invention may be established according to clinical trial protocol of which the following is an example.

### EXAMPLE 1

This is a parallel, double-blind, placebo-controlled study of 465 patients who meet the Rome II criteria for Irritable Bowel Syndrome.

Patients who meet entry criteria at Visits 1 and 2 will continue in a 1 week drug-free period where they will complete their symptom diaries and then will be randomized to one of the following three treatment groups: duloxetine 60 mg QD, duloxetine 60 mg BID or placebo. Randomization will be performed in a 1:1:1 ratio. Patients will be stratified into two groups: patients with a current major depressive disorder and patients without a current major depressive disorder. Following the screening phase, patients will be treated in a double-blind manner for 14 weeks, two of which will be used for titration and tapering.

The efficacy of duloxetine 60 mg QD and duloxetine 60 mg BID will be compared with placebo on the reduction of pain severity during a 12-week, double-blind, therapy phase in patients who have irritable bowel syndrome (IBS), with or without major depression. Pain severity will be assessed by using average daily pain severity scores from the patient IBS study diary. Efficacy of duloxetine may also be shown via improvement of one or all of the following measures:
Improvement in the Clinical Global Impression of Severity (CGI)
Improvement in the Patient Global Impression of Improvement (PGI)
Improvement in the Brief Pain Inventory (BPI)
Improvement in the Daily Patient Diary (via symptom description)
Improvement in the Irritable Bowel Syndrome Symptoms Assessment Scale (IBS-SAS)
Improvement in the Bristol Stool Scale

Improvement in symptoms of IBS via administration of duloxetine 60 mg QD and duloxetine 60 mg BID compared with placebo can be assessed via analysis of the results of implementation of this protocol in terms of discontinuation pattern, treatment-emergent adverse events, vital signs, electrocardiograms (ECG), and laboratory analysis.

1. (canceled)

2. (canceled)

3. (canceled)

4. A pharmaceutical composition for treating a gastrointestinal disorder in a patient, comprising duloxetine as the active ingredient with one or more pharmaceutically acceptable carriers.

5. A method of treating gastrointestinal disorder in a patient comprising administering to the patient in need thereof duloxetine.

6. The method of claim 5 wherein the disorder is:
inflammatory bowel disease (IBD),
functional bowel disorders (FBD),
dyspepsia,
iletis,
ischemic bowel disease,
irritable bowel syndrome,
gastroesophageal reflux or
diarrhea.

7. The method of claim 6 wherein the disorder is irritable bowel syndrome.
